(19) United States
(12) Patent Application Publication
(10) Pub. No.: US 2017/0340690 A1
(43) Pub. Date: Nov. 30, 2017

(54) METHOD FOR IMPROVING INTESTINAL HEALTH USING EXTRACTS OF CODONOPSIS LANCEOLATA

(71) Applicant: KOREA INSTITUTE OF SCIENCE AND TECHNOLOGY, Seoul (KR)

(72) Inventors: Cheol-Ho PAN, Gangneung-si (KR); Kyungsu KANG, Gangneung-si (KR); Kwang-Hyun CHA, Gangneung-si (KR); Eun Ha LEE, Gangneung-si (KR); Hee Ju LEE, Gangneung-si (KR); Song Yi KOO, Gangneung-si (KR); Yongsoo CHOI, Gangneung-si (KR)

(73) Assignee: KOREA INSTITUTE OF SCIENCE AND TECHNOLOGY, Seoul (KR)

(21) Appl. No.: 15/596,404

(22) Filed: May 16, 2017

(30) Foreign Application Priority Data
May 31, 2016 (KR) 10-2016-0067553

(51) Int. Cl. A61K 36/344; A23L 33/135; C12N 1/20; A61K 35/741; A61K 35/00

(52) U.S. Cl. CPC A61K 36/344; C12N 1/20; A61K 35/741; A23L 33/135; A23V 2002/00; A61K 2035/115

(57) ABSTRACT

Disclosed is a method for improving intestinal microflora, which includes administering an extract of Codonopsis lanceolata of an amount effective for improving intestinal microflora to a subject in need thereof. The extract can promote the proliferation of beneficial intestinal bacteria and suppress the proliferation of harmful bacteria. Also, the extract can improve intestinal health or intestinal function and improve defecation disorder. In addition, a synergic effect can be obtained when the composition of the present disclosure is used together with probiotics.

### CROSS-REFERENCE TO RELATED APPLICATION

This application claims the priority of Korean Patent Application No. 10-2016-0067553, filed on May 31, 2016, and all the benefits accruing therefrom under 35 U.S.C. §119, the contents of which in its entirety are herein incorporated by reference.

### BACKGROUND

### 1. Field

Disclosed in the present disclosure is a method for improving intestinal microflora, which includes administering an extract of Codonopsis lanceolata of an amount effective for improving intestinal microflora to a subject in need thereof. Also, Disclosed in the present disclosure is a method for cultivating intestinal microorganisms, which comprises cultivating the intestinal microorganisms in culture medium comprising an extract of Codonopsis lanceolata to promote the growth of intestinal microorganisms.

[Description about National Support Research and Development]

This research was conducted by the support of the Ministry of Agriculture, Food and Rural Affairs under the supervision of Seoul National University (Research management specialized agency: Korea Institute of Planning & Evaluation for Technology in Food, Agriculture, Forestry & Fisheries, research subject title: Development of materials and fermented milk products for improving human immune function through improvement of intestinal microflora, subject Identification No.: 315067-03).

### 2. Description of the Related Art

Recently, a close correlation between microorganisms in the human body, particularly intestinal microorganisms, and human diseases are being reported consistently. Accordingly, researches are continuously conducting in order to improve human health by using intestinal microorganisms. Especially, probiotics which are known to provide benefits for humans are being studied actively. Examples include methods for delivering lactic acid bacteria to the intestines or improving their survivability in the intestines so that the lactic acid bacteria known to be associated with intestinal health can act effectively.

### SUMMARY

The present disclosure is directed to improving intestinal microflora.

The present disclosure is also directed to promoting proliferation of beneficial intestinal bacteria and suppressing proliferation of harmful intestinal bacteria.

The present disclosure is also directed to enhancing the effect of probiotics.

The present disclosure is also directed to improving intestinal health or intestinal function.

In an aspect, the present disclosure provides a method for improving intestinal microflora, which includes administering an extract of Codonopsis lanceolata of an amount effective for improving intestinal microflora to a subject in need thereof.

In another aspect, the present disclosure provides a method for improving intestinal health or intestinal function, which includes administering an extract of Codonopsis lanceolata of an amount effective for improving intestinal health or intestinal function to a subject in need thereof.

In an exemplary embodiment, the extract of Codonopsis lanceolata may promote the proliferation or growth of beneficial intestinal bacteria or may suppress the proliferation or growth of harmful intestinal bacteria.

In an exemplary embodiment, the extract of Codonopsis lanceolata may suppress β-glucuronidase activity, suppress tryptophanase activity or increase intestinal short-chain fatty acids contents.

In an exemplary embodiment, the short-chain fatty acid may be one or more selected from a group consisting of acetate, propionate, isobutyrate, butyrate, isovalerate and valerate.

In an exemplary embodiment, the extract of Codonopsis lanceolata may improve defecation disorder.

In an exemplary embodiment, the extract of Codonopsis lanceolata may be administered together with probiotics.

In an exemplary embodiment, the extract of Codonopsis lanceolata may be administered in the form of a health food composition or a pharmaceutical composition.

In an exemplary embodiment, the health food composition may be a natural prebiotic composition.

In an exemplary embodiment, the extract of Codonopsis lanceolata may be contained in an amount of 0.1-100% based on total weight of the health food composition or the pharmaceutical composition, based on dry weight.

In an exemplary embodiment, the health food composition or the pharmaceutical composition may be administered orally with an administration dosage of 1-2000 mg/kg/day.

In an exemplary embodiment, the extract of Codonopsis lanceolata may be an extract of root of Codonopsis lanceolata.

In an exemplary embodiment, the extract may be a water extract, an organic solvent extract or an extract of aqueous solution containing organic solvents.

In an exemplary embodiment, the organic solvent may be an alcohol.

In an exemplary embodiment, the alcohol may be a C₁-C₅ lower alcohol.

In an exemplary embodiment, the C₁-C₅ lower alcohol may be ethanol or methanol.

In an exemplary embodiment, the concentration of the aqueous solution containing organic solvents may be 0.1%-99% (v/v).

In further another aspect, the present disclosure privides a method for cultivating intestinal microorganisms, which comprises cultivating the intestinal microorganisms in culture medium comprising an extract of Codonopsis lanceolata to promote the growth of intestinal microorganisms.

In an exemplary embodiment, the intestinal microorganisms may be beneficial intestinal bacteria.

In an exemplary embodiment, the intestinal microorganisms may be probiotic bacteria.

In an exemplary embodiment, the intestinal microorganisms may be lactic acid bacteria.

In an exemplary embodiment, the lactic acid bacteria may be one or more selected from a group consisting of Lactobacillus, Lactococcus, Leuconostoc, Pediococcus and Bifidobacterium.

In an aspect, the composition containing an extract of Codonopsis lanceolata of the present disclosure can promote the proliferation of beneficial intestinal bacteria, suppress the proliferation of harmful intestinal bacteria, increase intestinal short-chain fatty acids contents and improve intestinal microflora. Also, the composition of the present disclosure can improve intestinal health or intestinal function and improve constipation or defecation disorder. In addition, a synergic effect can be obtained when the composition of the present disclosure is used together with probiotics. The composition of the present disclosure containing an extract of Codonopsis lanceolata has no side effect on the human body because it is prepared from a natural product.

### BRIEF DESCRIPTION OF THE DRAWINGS

FIG. 1 shows a result of investigating the growth of intestinal microorganisms under a normal medium condition when fructooligosaccharide (FOS), chloramphenicol or an extract of Codonopsis lanceolata is administered.

FIG. 2 shows a result of investigating the growth of intestinal microorganisms under a high-protein diet simulation condition when fructooligosaccharide (FOS), chloramphenicol or an extract of Codonopsis lanceolata is administered.

FIG. 3 shows the activity of β-glucuronidase when fructooligosaccharide (FOS), chloramphenicol or an extract of Codonopsis lanceolata is administered.

FIG. 4 shows the activity of tryptophanase when fructooligosaccharide (FOS), chloramphenicol or an extract of Codonopsis lanceolata is administered.

### DETAILED DESCRIPTION

Codonopsis lanceolata (Siebold & Zucc.) Trautv., i.e. deodeok is a perennial climber belonging to the family Campanulaceae with edible root, stem and leaves. Deodeok is also used as a natural medicine with the name codonopsidis radix. Deodeok is known to have various medicinal effects. For example, it is known to be effective in treating bronchitis, cold, convulsion, neurosis, cancer, obesity, hyperlipidemia, edema, hepatitis, etc. However, nothing is known about the effect of deodeok or a deodeok extract on the improvement of intestinal microflora or improvement of intestinal function.

Hereinafter, the present disclosure is described in detail.

In an aspect, the present disclosure provides a method for improving intestinal microflora, which includes administering an extract of Codonopsis lanceolata of an amount effective for improving intestinal microflora to a subject in need thereof.

In the present disclosure, improvement of intestinal microflora may mean promotion of the growth of beneficial intestinal bacteria and suppression of the growth of harmful intestinal bacteria, with balance between the beneficial intestinal bacteria and the harmful intestinal bacteria.

In an exemplary embodiment, the extract of Codonopsis lanceolata may be administered in the form of a health food composition or a pharmaceutical composition.

In an exemplary embodiment, the extract of Codonopsis lanceolata may function as a prebiotic and the extract of Codonopsis lanceolata may be administered in the form of a prebiotic composition. The prebiotic composition which contains the extract of Codonopsis lanceolata provides an effect of enhancing the function of probiotics.

In an exemplary embodiment, the extract of Codonopsis lanceolata may be administered together with probiotics.

In the present disclosure, the prebiotic may mean an ingredient which is used by microorganisms including beneficial bacteria to provide beneficial effect for the health of a host by promoting the growth or activity of the microorganisms.

In the present disclosure, the probiotics may mean microorganisms which provide a good effect in the body.

In an exemplary embodiment, the extract of Codonopsis lanceolata may improve intestinal health or intestinal function.

In the present disclosure, the extract refers to a substance extracted from a natural product, regardless of the extraction method, extraction solvent or the type of the extract. It is used in a broad concept, including an extract obtained by otherwise processing or treating the extract. Specifically, the processing or treating the extract may be additionally fermenting or enzymatically treating the extract. Accordingly, in the present disclosure, the extract is used in a broad concept, including a fermentation product, a concentrate and a dried product.

In the present disclosure, a method for preparing the extract of Codonopsis lanceolata is not particularly limited. In the present disclosure, the extract of Codonopsis lanceolata may include a leachate obtained by leaching Codonopsis lanceolata, a concentrate obtained by concentrating all or part of the extract, an extract prepared by drying the concentrate or a chemical substance which is contained in the extract and exerts a main effect.

In an exemplary embodiment, when Codonopsis lanceolata is extracted with a solvent, it may be extracted by adding a solvent corresponding to about 1-15 times, specifically about 10 times, of the Codonopsis lanceolata, although not being limited thereto. The extraction may be hot extraction, cold extraction, reflux condensation extraction, ultrasonic extraction, etc. However, any extraction method obvious to those skilled in the art may be used without limitation. Although the extraction may be performed at room temperature, it may be performed at elevated temperatures for more effective extraction. The extraction may be performed at specifically about 40-100° C., more specifically about 80° C., although not being limited thereto. The extraction may be performed for specifically about 2-4 hours, more specifically about 3 hours. However, it may vary depending on extraction solvent, extraction temperature, etc. without being limited thereto. The extraction may be performed once or several times in order to obtain the active ingredient in a larger amount. Specifically, the extraction may be performed 1-5 times, more specifically 3 times, and the obtained extracts may be combined.

The Codonopsis lanceolata used in the present disclosure may be contained in the form of an extract as well as a herbal medicine itself, a pulverization product of the herbal medicine or a dried pulverization product of the herbal medicine, although not being limited thereto. In addition, the Codonopsis lanceolata used in the present disclosure is not limited as to how it is obtained. It may be either cultivated or purchased commercially.

In an exemplary embodiment, the extract of Codonopsis lanceolata may promote the proliferation or growth of beneficial intestinal bacteria and/or suppress the proliferation or growth of harmful intestinal bacteria.

In the present disclosure, the beneficial intestinal bacteria may collectively refer to microorganisms that inhabit in the intestines and provide beneficially effects for the human body. For example, the beneficial intestinal bacteria may include probiotics. For example, the beneficial intestinal bacteria may include Bifidobacterium, Lactobacillus, Lactococcus, Streptococcus, Akkermansia, Faecalibacterium or Enterococcus, although not being limited thereto.

Meanwhile, in the present disclosure, the harmful intestinal bacteria may collectively refer to microorganisms that inhabit in the intestines and provide harmful effects for the human body such as enteritis, etc. For example, the harmful intestinal bacteria may include Escherichia coli, Fusobacterium, Clostridium or Porphyromonas, although not being limited thereto.

In an aspect, the extract of Codonopsis lanceolata may suppress the activity of β-glucuronidase. β-Glucuronidase is a marker of harmful intestinal bacteria. Decreased activity of β-glucuronidase means that the growth of harmful intestinal bacteria is suppressed.

In this aspect, the extract of Codonopsis lanceolata may suppress the activity of tryptophanase. Tryptophanase is a marker of harmful intestinal bacteria. Decreased activity of tryptophanase means that the growth of harmful intestinal bacteria is suppressed.

In another aspect, the extract of Codonopsis lanceolata may increase intestinal short-chain fatty acids contents. In the present disclosure, the short-chain fatty acids may mean fatty acids with 6 or less carbon atoms. The short-chain fatty acid may be one or more selected from a group consisting of acetate, propionate, isobutyrate, butyrate, isovalerate and valerate. The short-chain fatty acid is produced from fermentation of dietary fiber (or saccharide) by good bacteria in the intestine. The short-chain fatty acid stimulates colon cells to inhibit inflammation and improve the intestinal structure, thereby preventing obesity and improving immunity function.

In this aspect, the extract of Codonopsis lanceolata may improve defecation disorder. The improvement of defecation disorder may include, for example, improvement of irregular defecation, constipation, diarrhea or dyschezia.

In an aspect, the extract of Codonopsis lanceolata may be contained in an amount of 0.1-100% based on total weight of the composition (e.g., a health food composition, a pharmaceutical composition or a prebiotic composition), based on dry weight.

The formulation of the health food composition according to an aspect of the present disclosure is not specially limited. For example, it may be formulated as a tablet, a granule, a powder, a liquid such as a drink, a caramel, a gel, a bar, etc. The food composition may contain various nutrients, vitamins, minerals (electrolytes), synthetic or natural flavorants, colorants, extenders (cheese, chocolate, etc.), pectic acid or salts thereof, alginic acid or salts thereof, organic acids, protective colloidal thickeners, pH control agents, stabilizers, antiseptics, glycerin, alcohols, carbonating agents used in carbonated drinks, etc. Each formulation of the food composition can be prepared by those skilled in the art without difficulty by mixing the active ingredient with ingredients commonly used in the art. A synergic effect may be achieved when the composition is used together with other substances, particularly a composition containing probiotics.

The pharmaceutical composition according to an aspect of the present disclosure may be in the form of various formulations for oral or parenteral administration. The formulations are prepared using a commonly used diluent or excipient such as a filler, an extender, a binder, a wetting agent, a disintegrant, a surfactant, etc. Solid formulations for oral administration include a tablet, a pill, a powder, a granule, a soft or hard capsule, etc. The solid formulation is prepared by mixing the active ingredient with one or more excipient, e.g., starch, calcium carbonate, sucrose, lactose, gelatin, etc. In addition to the simple excipient, a lubricant such as magnesium stearate, talc, etc. is also used. Liquid formulations for oral administration include a suspension, a liquid for internal use, an emulsion, a syrup. In addition to a commonly used simple diluent such as water and liquid paraffin, various excipients, e.g., a wetting agent, a sweetener, an aromatic, a preservative, etc. may also be contained.

In an exemplary embodiment, the composition may be administered parenterally or orally depending on purposes. The administration may be made once or several times a day with a dosage of 1-2000 mg/kg/day. The administration dosage for a particular patient may vary depending on the body weight, age, sex and health condition of the patient, diet, administration time, administration method, excretion rate, severity of a disease, etc.

The pharmaceutical composition according to an aspect of the present disclosure may be prepared into any type of pharmaceutically acceptable formulation including an oral formulation such as a powder, a granule, a tablet, a soft or hard capsule, a suspension, an emulsion, a syrup, an aerosol, etc., an injection, a sterile solution for injection, etc. according to common methods.

In an exemplary embodiment, the extract of Codonopsis lanceolata may include an extract of the aboveground part or underground part of Codonopsis lanceolata. The aboveground part of Codonopsis lanceolata may include the stem, leaf, flower and fruit of Codonopsis lanceolata and the underground part may include root. The extract of Codonopsis lanceolata may be an extract of one or more aboveground parts of Codonopsis lanceolata or an extract obtained from a mixture of the aboveground and underground parts of Codonopsis lanceolata.

In an exemplary embodiment, the extract may include a water extract, an organic solvent extract or an organic solvent aqueous solution extract. In an aspect, as the organic solvent, hexane, methylene chloride, an alcohol, etc. may be used, although not being limited thereto. The water includes distilled water or purified water and the organic solvent includes one or more selected from a group consisting of an alcohol such as a C₁-C₅ lower alcohol, acetone, ether, ethyl acetate, diethyl ether, methanol, ethyl methyl ketone and chloroform, although not being limited thereto.

In this aspect, the concentration of the organic solvent aqueous solution may be 0.1%-99% (v/v). For example, the concentration of the organic solvent aqueous solution may be 1% or higher, 5% or higher, 10% or higher, 20% or higher, 25% or higher, 50% or higher, 55% or higher, 60% or higher, 65% or higher, 70% or higher, 75% or higher, 80% or higher, 85% or higher, 90% or higher, 95% or higher or 97% or higher and 99% or lower, 95% or lower, 90% or lower, 85% or lower, 80% or lower, 75% or lower, 70% or lower, 65% or lower, 60% or lower, 55% or lower, 50% or lower, 45% or lower or 40% or lower, although not being limited thereto. For example, it may be an 85-99% ethanol extract.

In an exemplary embodiment, the extract may be a 95% ethanol extract.

In an aspect, the present disclosure provides a kit for improving intestinal health, which includes the composition containing an extract of Codonopsis lanceolata and an instruction.

The instruction may instruct oral administration of the composition with an administration dosage of 1-2000 mg/kg/day. Also, in this aspect, the instruction may instruct coadministration with a composition containing probiotics.

The composition containing probiotics may be a health food composition or pharmaceutical composition containing probiotics. For example, it may be a fermented milk product containing lactic acid bacteria.

In the present disclosure, the coadministration includes administration of a composition containing an extract of Codonopsis lanceolata and a composition containing probiotics at the same time or administration of one followed by administration of the other.

In an exemplary embodiment, the composition containing an extract of Codonopsis lanceolata as an active ingredient and the composition containing probiotics may be administered to a subject at the same time.

In further another aspect, the present disclosure privides a method for cultivating intestinal microorganisms, which comprises cultivating the intestinal microorganisms in culture medium comprising an extract of Codonopsis lanceolata to promote the growth of intestinal microorganisms.

In an exemplary embodiment, the intestinal microorganisms may be beneficial intestinal bacteria.

In an exemplary embodiment, the intestinal microorganisms may be probiotic bacteria.

In an exemplary embodiment, the intestinal microorganisms may be lactic acid bacteria.

In an exemplary embodiment, the lactic acid bacteria may be one or more selected from a group consisting of Lactobacillus, Lactococcus, Leuconostoc, Pediococcus and Bifidobacterium.

Hereinafter, the present disclosure will be described in detail through examples and test examples. However, the following examples and test examples are for illustrative purposes only and it will be apparent to those of ordinary skill in the art that the scope of the present disclosure is not limited by the examples and test examples.

### [Example 1] Preparation of an Extract of Codonopsis lanceolata

The root of Codonopsis lanceolata purchased from a farmhouse (Pyeongchang, Gangwon-do, Korea) was washed cleanly with water, pulverized to a suitable size without peeling the skin and extracted once in an extractor at room temperature for 24 hours by adding 2.0 kg of the Codonopsis lanceolata and 3 L of 95% ethanol. The extract was filtered through filter paper and the solvent was removed under reduced pressure. 85.49 g of a 95% ethanol extract was obtained.

### [Test Example 1] Securing of Human Fecal Sample

For culturing of intestinal microorganisms, 10-30 g of fecal samples were secured from seven healthy young Koreans who had not been prescribed antibiotics for 3 months. The samples were transfered to an anaerobic workstation within 30 minutes and treated quickly. After adding glycerol as an additive for storage at low temperature, the mixtures were stored in an ultra-low temperature freezer with 40 mL each and were used as samples for culturing of intestinal microorganisms.

### [Test Example 2] Anaerobic Continuous Culturing of Intestinal Microorganisms

Anaerobic continuous culturing was conducted using a bioreactor to maintain the culturing condition of intestinal microorganisms stably. A basal medium described in Table 1 was used for the culturing and a medium described in Table 2 was used for a high-protein diet simulation group.

TABLE 1
Composition of basal medium

| Ingredients | Contents (g/L) |
|---|---|
| Peptone | 1.3 |
| Yeast extract | 2 |
| NaHCO₃ | 2 |
| L-Cysteine-HCl | 0.5 |
| Bile salt | 0.5 |
| Hemin | 0.005 |
| NaCl | 0.8 |
| KH₂PO₄ | 0.04 |
| K₂HPO₄ | 0.04 |
| MgSO₄ | 0.01 |
| CaCl₂ | 0.01 |
| Mucin | 4 |
| Arabinogalactan | 5 |
| Starch | 5 |
| Vitamin K₁ | 10 (μL) |

TABLE 2
Composition of high-protein diet simulation medium

| Ingredients | Contents (g/L) |
|---|---|
| Peptone | 1.3 |
| Yeast extract | 2 |
| NaHCO₃ | 2 |
| L-Cysteine-HCl | 0.5 |
| Bile salt | 0.5 |
| Hemin | 0.005 |
| NaCl | 0.8 |
| KH₂PO₄ | 0.04 |
| K₂HPO₄ | 0.04 |
| MgSO₄ | 0.01 |
| CaCl₂ | 0.01 |
| Mucin | 4 |
| Casein | 8 |
| Starch | 2 |
| Vitamin K₁ | 10 (μL) |

Each bioreactor containing 300 mL of a medium was sterilized and maintained at an anaerobic condition by injecting nitrogen gas. Then, the sample of intestinal microorganisms prepared in Test Example 1 was inoculated. After conducting batch culturing for 24 hours, continuous culturing was performed using a medium of the same composition. Total residence time was 24 hours. Culturing temperature was maintained at 37° C. and pH was maintained at 5.5 using a 1 N hydrochloric acid solution and a 1 N sodium hydroxide solution. Samples were taken from the culture once a day and subjected to short-chain fatty acid (SCFA) analysis for investigation of stabilization. The culturing condition was maintained for at least 30 days.

### [Test Example 3] Investigation of Improvement of Intestinal Microflora/Defecation Function

In order to quickly evaluate the effect of the candidate materials on improvement of intestinal microflora, culturing was performed using a 96-well plate. 10 μL of the culture medium of intestinal microorganisms maintained stably through anaerobic continuous culturing was inoculated to 1 mL of a medium for investigation described in Table 3. Culturing was performed at 37° C. for 48 hours by treating an extract of Codonopsis lanceolata for evaluating the effect of improving intestinal microflora at a concentration of 100 μg/mL (0.2% DMSO). As a positive control group, fructooligosaccharide (FOS, 1 mg/mL) which is a prebiotic well known to be effective in improving intestinal microflora was used. At the same time, chloramphenicol (10 or 40 μg/mL) which is an antibiotic known to worsen intestinal microflora was used to compare the effect of improving intestinal microflora. Then, the effect of improving intestinal microflora was investigated by measuring the growth of intestinal microorganisms, the enzymatic activity of harmful bacteria, the content of short-chain fatty acids, etc. using the culture medium of microorganisms.

TABLE 3
Composition of medium for investigation

| Ingredients | Contents (g/L) |
|---|---|
| Peptone | 1.3 |
| Yeast extract | 2 |
| NaHCO₃ | 2 |
| L-Cysteine-HCl | 0.5 |
| Bile salt | 0.5 |
| Hemin | 0.005 |
| NaCl | 0.8 |
| KH₂PO₄ | 0.04 |
| K₂HPO₄ | 0.04 |
| MgSO₄ | 0.01 |
| CaCl₂ | 0.01 |
| Mucin | 0.8 |
| Arabinogalactan | 1 |
| Starch | 1 |
| Vitamin K₁ | 10 (μL) |

### [Test Example 3-1] Measurement of Growth of Intestinal Microorganisms

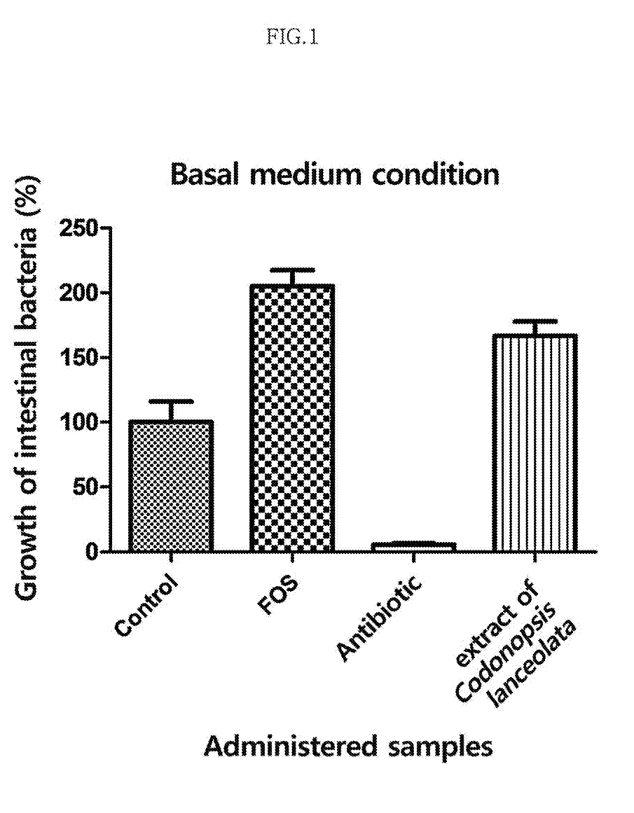

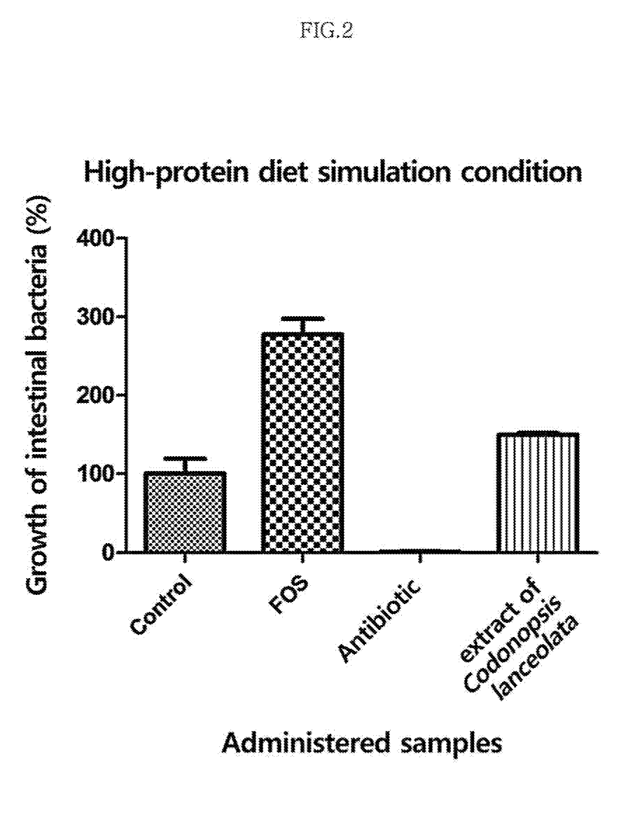

100 μL of the culture medium treated with fructooligosaccharide (FOS), an antibiotic or an extract of Codonopsis lanceolata was transferred to a 96-well plate and the relative growth of microorganisms was measured by measuring absorbance at 600 nm using a plate reader. As a result, it was confirmed that the extract of Codonopsis lanceolata increases the growth of intestinal microorganisms. Specifically, whereas fructooligosaccharide (FOS) which is a prebiotic having a proven effect of improving intestinal microflora increased the growth of intestinal microorganisms, the administration of the antibiotic (chloramphenicol) known to worsen intestinal microflora resulted in decreased growth of intestinal microorganisms. When the extract of Codonopsis lanceolata was administered, the growth of intestinal microorganisms was increased. Accordingly, it was confirmed that the extract of Codonopsis lanceolata is effective in improving intestinal microflora. The effect of the extract of Codonopsis lanceolata of promoting the growth of intestinal microflora was confirmed not only under the normal medium condition (FIG. 1) but also under the high-protein diet simulation condition (FIG. 2).

### [Test Example 3-2] Measurement of β-Glucuronidase Activity

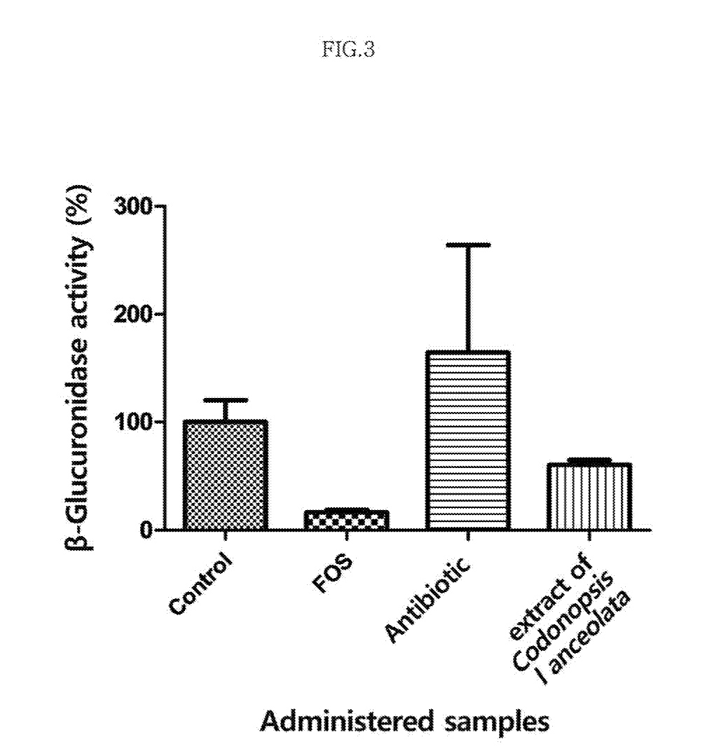

The activity of β-glucuronidase was measured using the culture medium of intestinal microorganisms treated with fructooligosaccharide (FOS), an antibiotic or an extract of Codonopsis lanceolata. The measurement of β-glucuronidase activity is a standard test in evaluation of intestinal health. The culture medium of intestinal microorganisms incubated and maintained with a culture medium in a fermenter under an anaerobic condition. 1.0 mL of the anaerobic culture medium was treated with fructooligosaccharide (FOS), an antibiotic or an extract of Codonopsis lanceolata and then 10 μL of the culture medium of intestinal microorganisms was inoculated. After incubation in an incubator at 37° C. for 48 hours under an anaerobic condition, the culture medium was used as an enzyme solution sample. After adding 20 μL of 20 mM 4-nitrophenyl-β-D-glucuronide to a 96-well plate and then adding 80 μL of the enzyme solution, reaction was performed at 37° C. for 60 minutes. After completing the reaction by adding 100 μL of 0.5 N NaOH, centrifugation was performed at 3500 rpm for 10 minutes. The supernatant was taken and absorbance was measured at 405 nm. Then, relative β-glucuronidase activity (%) was calculated by dividing the enzymatic activity by the degree of growth of intestinal microorganisms (OD₆₀₀). As a result, it was confirmed that, whereas fructooligosaccharide (FOS) which is a prebiotic with a proven effect of improving intestinal microflora reduces β-glucuronidase activity, the administration of the antibiotic (chloramphenicol) known to worsen intestinal microflora resulted in increased β-glucuronidase activity in the culture medium of intestinal microorganisms. When the extract of Codonopsis lanceolata was administered, the β-glucuronidase activity was decreased. As a result, it was confirmed that the extract of Codonopsis lanceolata is effective in improving intestinal microflora (FIG. 3).

### [Test Example 3-3] Measurement of Tryptophanase Activity

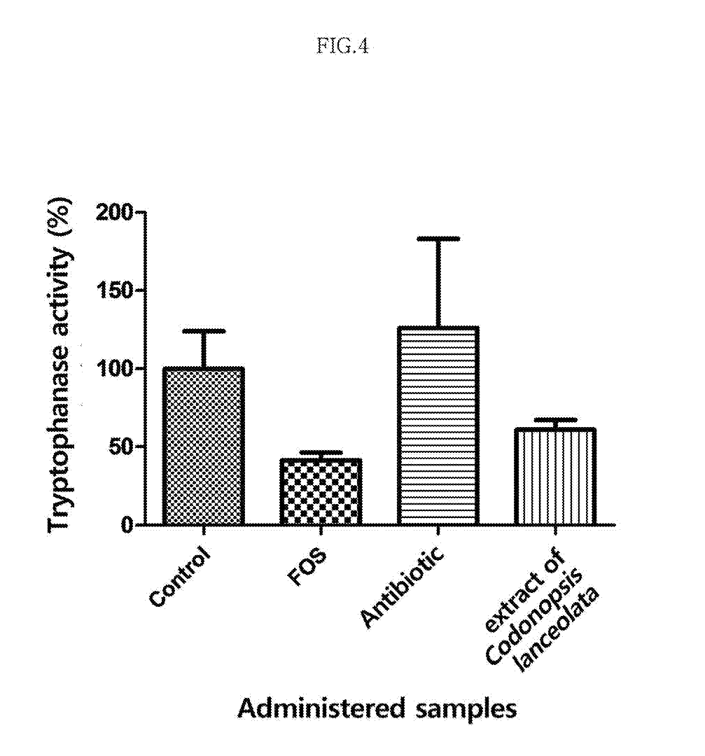

Tryptophanase activity was measured using the culture medium of intestinal microorganisms with fructooligosaccharide (FOS), an antibiotic or an extract of Codonopsis lanceolata. The measurement of tryptophanase activity is a standard test in evaluation of intestinal health. The culture medium of intestinal microorganisms incubated and maintained with a culture medium in a fermenter under an anaerobic condition. 1.0 mL of the anaerobic culture medium was treated with a natural product sample and then 10 μL of the culture medium of intestinal microorganisms was inoculated. After incubation in an incubator at 37° C. for 48 hours under an anaerobic condition, the culture medium was used as an enzyme solution sample. A reaction mixture solution was prepared by dissolving 2.75 mg of pyridoxal phosphate, 19.6 mg of disodium EDTA dehydrate and 10 mg of bovine serum albumin in 100 mL of a 0.05 M potassium phosphate buffer (pH 7.5). 40 μL of the reaction mixture solution, 20 μL of the culture sample and 40 μL of 40 mM tryptophan were mixed well in a 96-well plate and reacted at 37° C. for 60 minutes. After completing the reaction by adding 100 μL of a color reagent (14.7 g of p-dimethylaminobenzaldehyde, 948 mL of 95% ethanol, 52 mL of H₂SO₄), centrifugation was performed at 3500 rpm for 10 minutes. The supernatant was taken and absorbance was measured at 550 nm. Then, relative tryptophanase activity (%) was calculated by dividing the enzymatic activity by the degree of growth of intestinal microorganisms (OD₆₀₀). As a result, it was confirmed that, whereas fructooligosaccharide (FOS) which is a prebiotic with a proven effect of improving intestinal microflora reduces tryptophanase activity, the administration of the antibiotic (chloramphenicol) known to worsen intestinal microflora resulted in increased tryptophanase activity in the culture medium of intestinal microorganisms. When the extract of Codonopsis lanceolata was administered, the β-glucuronidase activity was decreased. As a result, it was confirmed that the extract of Codonopsis lanceolata is effective in improving intestinal microflora (FIG. 4).

### [Test Example 3-4] Analysis of Content of Short-Chain Fatty Acids

Gas chromatographic (GC) analysis was performed to analyze the content of short-chain fatty acids (SCFAs) in the culture medium of intestinal microorganisms treated with fructooligosaccharide (FOS), an antibiotic or an extract of Codonopsis lanceolata. The analysis sample was subjected to centrifugation of the 96-well plate at 3500 rpm for 10 minutes. 400 μL of the obtained supernatant was stabilized by adding 20 μL of 50% sulfuric acid and then 40 μL of an internal standard (1% 2-methylpentanoic acid) was added. Then, after adding 400 μL of ethyl ether, the mixture was mixed for about 2 minutes by vortexing. The sample was centrifuged and the top ethyl ether layer was recovered and subjected to GC analysis. The analysis was performed using Bruker 450-GC and the Supelco's Nukol Fused silica capillary column (30 mm×0.25 mm×0.25 μm). The analysis condition is described in Table 4.

TABLE 4
Gas chromatographic analysis condition

| | Experimental condition |
|---|---|
| Injector | 225° C. |
| Oven | Initial 110° C., 5.5° C./min, 15 min |
| Detector | FID, 225° C. |
| Carrier gas | 2.0 mL/min, helium flow 30 mL/min |
| injection | 2 μL, 3:1 split |

As a result, the content of short-chain fatty acids in the non-treated control group was measured to be 18.6 mM. In contrast, the treatment with fructooligosaccharide (FOS) which is a prebiotic with a proven effect of improving intestinal microflora increased the content of short-chain fatty acids to 25.41 mM. When the antibiotic (chloramphenicol) known to worsen intestinal microflora was administered, the content of short-chain fatty acids was greatly decreased to 2.03 mM. When the extract of Codonopsis lanceolata was administered, the content of short-chain fatty acids was increased to 19.28 mM. Accordingly, it was confirmed that the extract of Codonopsis lanceolata is effective in improving intestinal microflora (Table 5).

TABLE 5
Change in content of short-chain fatty acids when fructooligosaccharide
(FOS), chloramphenicol or extract of Codonopsis lanceolata was administered

| | Composition of short-chain fatty acids (%) | | | | | | Content |
|---|---|---|---|---|---|---|---|
| | Acetate | Propionate | Isobutyrate | Butyrate | Isovalerate | Valerate | (mM) |
| Control | 46.25 ± 4.32 | 15.36 ± 0.10 | 4.35 ± 0.55 | 25.06 ± 1.76 | 8.28 ± 0.44 | 0.71 ± 0.04 | 18.60 ± 1.21 |
| Fructooligosaccharide | 46.44 ± 1.79 | 23.54 ± 0.33 | 2.97 ± 0.54 | 21.57 ± 1.37 | 5.48 ± 0.25 | Not | 25.41 ± 0.55 |
| (FOS) | | | | | | detected | |
| Antibiotic | 54.36 ± 3.23 | 25.40 ± 0.89 | Not | 20.24 ± 0.64 | Not | Not | 2.03 ± 0.06 |
| (chloramphenicol) | | | detected | | detected | detected | |
| extract of | 45.87 ± 1.16 | 16.79 ± 1.16 | 4.24 ± 0.84 | 24.21 ± 2.30 | 8.06 ± 0.59 | 0.83 ± 0.002 | 19.28 ± 1.16 |
| Codonopsis | | | | | | | |
| lanceolata | | | | | | | |

What is claimed is:

1. A method for improving intestinal microflora, which comprises administering an extract of Codonopsis lanceolata of an amount effective for improving intestinal microflora to a subject in need thereof.

2. The method for improving intestinal microflora according to claim 1, wherein the extract of Codonopsis lanceolata promotes the proliferation or growth of beneficial intestinal bacteria or suppresses the proliferation or growth of harmful intestinal bacteria.

3. The method for improving intestinal microflora according to claim 1, wherein the extract of Codonopsis lanceolata suppresses β-glucuronidase activity, suppresses tryptophanase activity or increases intestinal short-chain fatty acids contents.

4. The method for improving intestinal microflora according to claim 3, wherein the short-chain fatty acid is one or more selected from a group consisting of acetate, propionate, isobutyrate, butyrate, isovalerate and valerate.

5. The method for improving intestinal microflora according to claim 1, wherein the extract of Codonopsis lanceolata improves defecation disorder.

6. The method for improving intestinal microflora according to claim 1, wherein the extract of Codonopsis lanceolata is administered together with probiotics.

7. The method for improving intestinal microflora according to claim 1, wherein the extract of Codonopsis lanceolata is administered in the form of a health food composition or a pharmaceutical composition.

8. The method for improving intestinal microflora according to claim 7, wherein the health food composition is a prebiotic composition.

9. The method for improving intestinal microflora according to claim 7, wherein the extract of Codonopsis lanceolata is comprised in an amount of 0.1-100% based on total weight of the health food composition or the pharmaceutical composition, based on dry weight.

10. The method for improving intestinal microflora according to claim 7, wherein the health food composition or the pharmaceutical composition is administered orally with an administration dosage of 1-2000 mg/kg/day.

11. The method for improving intestinal microflora according to claim 1, wherein the extract of Codonopsis lanceolata is an extract of root of Codonopsis lanceolata.

12. The method for improving intestinal microflora according to claim 1, wherein the extract is a water extract, an organic solvent extract or an extract of aqueous solution containing organic solvents.

13. The method for improving intestinal microflora according to claim 12, wherein the organic solvent is an alcohol.

14. The method for improving intestinal microflora according to claim 13, wherein the alcohol is a C₁-C₅ lower alcohol.

15. The method for improving intestinal microflora according to claim 14, wherein the C₁-C₅ lower alcohol is ethanol or methanol.

16. The method for improving intestinal microflora according to claim 12, wherein the concentration of the aqueous solution containing organic solvents is 0.1%-99% (v/v).

17. Method for cultivating intestinal microorganisms, which comprises cultivating the intestinal microorganisms in culture medium comprising an extract of Codonopsis lanceolata to promote the growth of intestinal microorganisms.

18. The method for cultivating intestinal microorganisms according to claim 17, wherein the intestinal microorganisms are probiotic bacteria.

19. The method for cultivating intestinal microorganisms according to claim 17, wherein the intestinal microorganisms are lactic acid bacteria.

20. The method for cultivating intestinal microorganisms according to claim 19, wherein the lactic acid bacteria is one or more selected from a group consisting of Lactobacillus, Lactococcus, Leuconostoc, Pediococcus and Bifidobacterium.
